(19) United States
(12) Patent Application Publication
(10) Pub. No.: US 2016/0213688 A1
(43) Pub. Date: Jul. 28, 2016

(54) INHALABLE MEDICAMENT

(71) Applicant: Teva Branded Pharmaceutical Products R&D, Inc., Frazer, PA (US)

(72) Inventor: Gokul H. Gopalan, Bethesda, MD (US)

(21) Appl. No.: 15/003,289

(22) Filed: Jan. 21, 2016

Related U.S. Application Data
(60) Provisional application No. 62/107,178, filed on Jan. 23, 2015.

(51) Int. Cl. A61K 31/573; A61K 9/12; A61M 15/00; A61K 9/00

(52) U.S. Cl. CPC A61K 31/573; A61K 9/008; A61K 9/124; A61M 15/009

(57) ABSTRACT

The present invention provides an inhalable pharmaceutical solution aerosol comprising beclometasone dipropionate, ethanol and a propellant selected from 1,1,1,2-tetrafluoroethane, 1,1,1,2,3,3,3-heptafluoropropane and a mixture thereof, wherein the aerosol has a droplet size having a mass median aerodynamic diameter of 0.5-2.0 μm, for use in the treatment of asthma in an individual with impaired hand-inhalation coordination.

### CROSS-REFERENCE TO RELATED APPLICATION

This application claims priority to U.S. Provisional Application No. 62/107,178, filed Jan. 23, 2015, the entire disclosure of which is incorporated herein by reference in its entirety for all purposes.

### FIELD OF THE INVENTION

The present invention relates to an inhalable medicament for use in the treatment of asthma, and more specifically to a medicament containing beclometasone dipropionate.

### BACKGROUND OF THE INVENTION

Asthma is a common inflammatory disease in which the airways of the respiratory system become chronically narrowed and constricted by oedema (fluid retention). During an attack, a patient suffers from laboured breathing accompanied especially by wheezing and coughing and by a sense of constriction in the chest due to bronchospasm (sudden constriction of the muscles in the walls of the bronchioles), mucosal oedema and mucus formation. Asthma is triggered by hyperreactivity to various stimuli (such as allergens or a rapid change in air temperature). In sensitised individuals, inhaled allergens (allergy triggers) provoke a hyperimmune response characterised by recruitment of immune cells and production of immunoglobulin E (IgE) antibodies. Asthma is caused by the attachment of IgE antibodies to mast cells (a resident cell of several types of tissues throughout the body, particularly in proximity to surfaces that interface the external environment). This attachment activates mast cells and on renewed exposure to the same antigen, degranulation of the mast cells occurs, leading to the rapid release of inflammatory mediators, such as histamine, proteoglycans, and cytokines. Asthma is a result of localised release of such mediators.

In a patient suffering from asthma, airflow obstruction is largely reversible and the patient experiences a significant response to inhaled bronchodilators and inhaled anti-inflammatories.

Asthma is generally treated using a combination of long-term treatment and short-term episodic treatment of acute attacks. Long-term treatment of asthma involves the use of anti-inflammatories and long-acting bronchodilators. Short-term episodic treatment employs short-acting bronchodilators. Inhaled glucocorticosteroids (ICS) are a feature of the currently preferred treatment for moderate to severe allergic asthma, and have been shown to act by suppressing the adaptive immune response while not suppressing innate immune response. There are various effective ICSs available in the art, for example, fluticasone propionate or beclometasone.

For example, the Flixotide Evohaler® marketed by Allen & Hanburys Ltd is a metered dose inhaler (MDI) containing fluticasone propionate as a pressurised suspension in the propellant HFA 134a. Fluticasone propionate is named as S-(fluoromethyl)-6α,9-difluoro-11β, 17-dihydroxy-16α-methyl-3-oxoandrosta-1,4-diene-17β-carbothioate, 17-propanoate.

A disadvantage of MDIs is that some patients have difficulty co-ordinating actuation of the inhaler with simultaneous inhalation. Some children, some of the elderly and some of those suffering from a joint disorder such as arthritis encounter this difficulty. A difficulty in co-ordinating actuation with inhalation is characterised by a larger positive or negative time difference between the start of an inhalation and actuation of a dose (TsIn). See Azouz et al., Journal of Aerosol Medicine and Pulmonary Drug Delivery, June 2014, 27 (3). pp. 193-199.

Such sub-optimal use can result in oropharyngeal impaction, increasing risk of local side effects and reducing the dose that reaches the lungs.

In order to address this problem, the use of a spacer is typically recommended for such patients. A spacer is an add-on device used to increase the ease of administering aerosolized medication from a metered-dose inhaler (MDI). The spacer adds space in the form of a tube or “chamber” between the canister of medication and the patient's mouth, allowing the patient to inhale the medication by breathing in slowly and deeply for five to 10 breaths. Spacers slow down the speed of the aerosol coming from the inhaler, meaning that less of the asthma drug impacts on the back of the mouth and somewhat more may get into the lungs. Because of this, less medication is needed for an effective dose to reach the lungs, and there are fewer side effects from corticosteroid residue in the mouth.

However, spacers are typically bulky and therefore reduce the portability of the patient's treatment. This is likely to have a negative impact on patient compliance. It is also necessary to clean the spacer frequently.

Despite these drawbacks, the use of a spacer is recommended in the brand owner's SmPC for Flixotide Evohaler®: “Flixotide Evohaler may be used with a Volumatic spacer device by patients who find it difficult to synchronise aerosol actuation with inspiration of breath.”

Another ICS used in the treatment of asthma is beclometasone dipropionate (INN), also known as beclomethasone dipropionate (USAN) or (8S,9R,10S,11S,13S,14S,16S,17R)-9-chloro-11-hydroxy-10,13,16-trimethyl-3-oxo-17-[2(propionyloxy)acetyl]-6,7,8,9,10,11,12,13,14,15,16,17-dodecahydro-3H-cyclopenta[a]phenanthren-17-yl propionate (IUPAC), and is the subject of the present invention.

Formulations of beclometasone dipropionate are known in the art. For example, U.S. Pat. No. 5,776,432 discloses a pharmaceutical solution aerosol formulation comprising beclometasone dipropionate, a hydrofluorocarbon propellant selected from 1,1,1,2-tetrafluoroethane (norflurane or propellant 134a), 1,1,1,2,3,3,3-heptafluoropropane (propellant 227) and a mixture thereof, and ethanol (anhydrous) to solubilise the beclometasone dipropionate in the propellant. The beclometasone dipropionate is dissolved in the formulation, and the formulation is substantially free of surfactant.

There is therefore the need for an ICS formulation which overcomes the above-mentioned problems.

### BRIEF SUMMARY OF THE INVENTION

Surprisingly, the inventors of the present application have found that a particular ICS MDI formulation is effective in the treatment of asthma even without the use of a spacer.

Accordingly, the present invention provides an inhalable pharmaceutical solution aerosol comprising beclometasone dipropionate, ethanol and a propellant selected from 1,1,1,2-tetrafluoroethane, 1,1,1,2,3,3,3-heptafluoropropane and a mixture thereof, wherein the aerosol has a droplet size having a mass median aerodynamic diameter of 0.5-2.0 μm, for use in the treatment of asthma in an individual with impaired hand-inhalation coordination.

Also provided by the present invention is a method for treating asthma in an individual with impaired hand-inhalation coordination, wherein the method comprises administering to the individual via inhalation an inhalable pharmaceutical solution aerosol comprising beclometasone dipropionate, ethanol and a propellant selected from 1,1,1,2-tetrafluoroethane, 1,1,1,2,3,3,3-heptafluoropropane and a mixture thereof, wherein the aerosol has a droplet size having a mass median aerodynamic diameter of 0.5-2.0 μm.

A pressurized metered-dose inhaler useful for treatment of asthma in an individual with impaired hand-inhalation coordination is additionally provided by the present invention, the pressurized metered-dose inhaler comprising a canister and an actuator having a discharge nozzle having an orifice diameter of 100-300 μm, wherein the canister has a vial containing a pharmaceutical solution comprising beclometasone dipropionate, ethanol and a propellant selected from 1,1,1,2-tetrafluoroethane, 1,1,1,2,3,3,3-heptafluoropropane or a mixture thereof, and wherein the pressurized metered-dose inhaler is configured to form, upon actuation, an inhalable aerosol of the pharmaceutical solution having a droplet size having a mass median aerodynamic diameter of 0.5-2.0 μm.

Impaired hand-inhalation coordination is characterised by a larger positive or negative time difference between the start of an inhalation and actuation of a dose (TsIn). Typically, the individual demonstrates either a positive or negative TsIn of greater than 0.2 seconds. In an embodiment, the individual demonstrates either a positive or negative TsIn of greater than 0.5 seconds. In a further embodiment, the individual demonstrates either a positive or negative TsIn of greater than 1.0 seconds. In a further embodiment, the individual demonstrates either a positive or negative TsIn of greater than 2.5 seconds.

TsIn is “positive” if actuation occurs after the start of an inhalation and “negative” if actuation occurs before the start of an inhalation. TsIn will be zero (0) when actuation occurs at the same time as the start of an inhalation.

### BRIEF DESCRIPTION OF THE DRAWINGS

FIG. 1 shows a study design of a historical real-life database analysis of children with asthma initiating their asthma therapy as either extra fine beclometasone dipropionate (BDP) via metered dose inhaler (MDI) according to the present invention or fluticasone propionate (FP) via MDI using a spacer device;

FIG. 2 shows the study population;

FIG. 3 shows the outcome of severe exacerbation rates in the study; and

FIG. 4 shows the odds ratios during the outcome year of the study.

### DETAILED DESCRIPTION OF CERTAIN EMBODIMENTS OF THE INVENTION

The active ingredient beclometasone dipropionate is generally present in a formulation in a therapeutically effective amount, i.e. an amount such that metered volumes of the medicament administered to the patient contains an amount of drug effective to exert the intended therapeutic action. The aerosol solution preferably contains 0.02 to 0.6 percent by weight, more preferably 0.05 to 0.5 percent by weight of beclometasone dipropionate, based on the total weight of the solution.

Ethanol is present in an amount effective to solubilise the beclometasone dipropionate in the propellant. Preferably, the solution contains 1 to 20 percent by weight of ethanol, more preferably 2 to 12 percent by weight and most preferably 4 to 10 percent by weight, based on the total weight of the aerosol solution. The ethanol will be present in an amount sufficient to dissolve substantially all of the medicament present in the formulation and to maintain the medicament dissolved over the time period and conditions experienced by commercial aerosol products. Preferably the ethanol is present in an amount to prevent precipitation of the active ingredient even at temperatures down to −20° C. The ethanol is preferably anhydrous ethanol, although trace amounts of water absorbed by the ingredients, for example during manufacture of the medicament, may be tolerated.

The hydrofluorocarbon propellant may be propellant 134a (1,1,1,2-tetrafluoroethane), propellant 227 (1,1,1,2,3,3,3-heptafluoropropane) or a mixture thereof. The solution preferably contains 80 to 99 percent by weight of propellant, more preferably 88 to 98 percent by weight, and most preferably 90 to 95 percent by weight, based on the total weight of the aerosol solution. The hydrofluorocarbon propellant is preferably the only propellant present in the formulations of the invention.

The solution of the present invention is preferably substantially free of surfactant. Surfactants are often added to suspensions to stabilise the suspension. However, since the formulation of the present invention is a solution, a surfactant is not required. Nevertheless, small quantities can be tolerated without adversely affecting the formulation. Preferably the formulation contains no more than 0.0005 percent by weight of a surfactant based on the total weight of the solution. Preferred formulations contain no surfactant. Presence of a significant amount of a surfactant is believed to be undesirable for solution formulations of beclometasone dipropionate because surfactants such as oleic acid and lecithin are believed to promote chemical degradation of the active ingredient when the latter is dissolved in the mixture of the propellant and ethanol.

A preferred solution according to the present invention comprises 0.02 to 0.6 percent by weight beclometasone dipropionate, 1 to 20 percent by weight ethanol and 80 to 99 percent by weight of propellant, wherein the percentages by weight are based on the total weight of the solution aerosol. A particularly preferred solution consists essentially of beclometasone dipropionate, ethanol and a propellant selected from 1,1,1,2-tetrafluoroethane, 1,1,1,2,3,3,3-heptafluoropropane and a mixture thereof; more preferably the solution consists of these components.

The solution of the present invention may be prepared by dissolving the desired amount of beclometasone dipropionate in the desired amount of ethanol accompanied by stirring or sonication. An aerosol container may then be filled using conventional cold-fill or pressure-fill methods.

The solution aerosol of the present invention is administered from a pressurised metered-dose inhaler (pMDI). A pMDI has two key components, namely a canister and an actuator (or mouthpiece). The canister has a vial for storing the solution coupled to a metering dose valve having an actuating stem. The container is housed in an actuator where the actuating step is in fluid communication with a discharge nozzle in the actuator. Actuation of the device releases a single metered dose of solution aerosol. The aerosol passes through the discharge nozzle resulting in a breaking up of the volatile propellant into droplets, followed by rapid evaporation of these droplets as they are inhaled into the lungs. The discharge nozzle preferably has an orifice diameter of 100-300 μm, more preferably 150-250 μm and most preferably 248 μm. This orifice size encourages the formation of droplets of the required size in the aerosol solution of the present invention.

The solution of the present invention when administered from the pMDI forms a fine aerosol of droplets which has a droplet size having a mass median aerodynamic diameter of 0.5-2.0 μm and preferably 0.8-1.2 μm. This small droplet size facilitates deep lung penetration. The droplet size may be measured using conventional techniques, such as using a Next Generation Pharmaceutical Impactor (NGI) or an Andersen eight-stage impactor (ACI).

Individuals with impaired hand-inhalation coordination can be found within the following groups: those aged from 5 to 11 years, those aged from 60 years and above, and those suffering from arthritis.

The present invention is effective for individuals who do not inhale with the use of a spacer, although the use of a spacer is not excluded.

The aerosol is particularly effective in treating asthma in individuals showing insufficient response to an inhalable formulation containing fluticasone propionate and HFA propellant providing a droplet size having a mass median aerodynamic diameter of greater than 2.0 μm.

The present invention will now be described with reference to the accompanying drawings, in which:

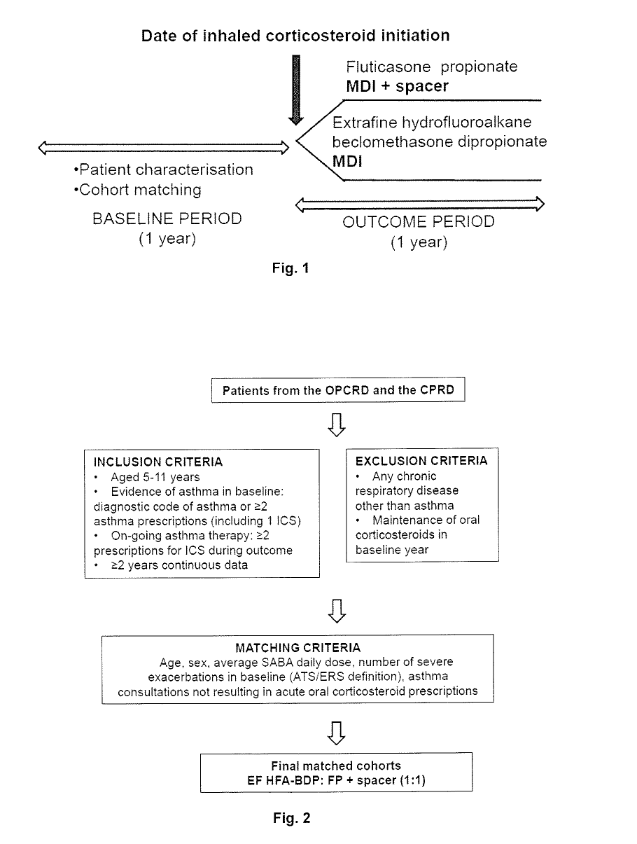

FIG. 1 shows a study design of a historical real-life database analysis of children with asthma initiating their asthma therapy as either extra fine beclometasone dipropionate (BDP) via metered dose inhaler (MDI) according to the present invention or fluticasone propionate (FP) via MDI using a spacer device;

FIG. 2 shows the study population;

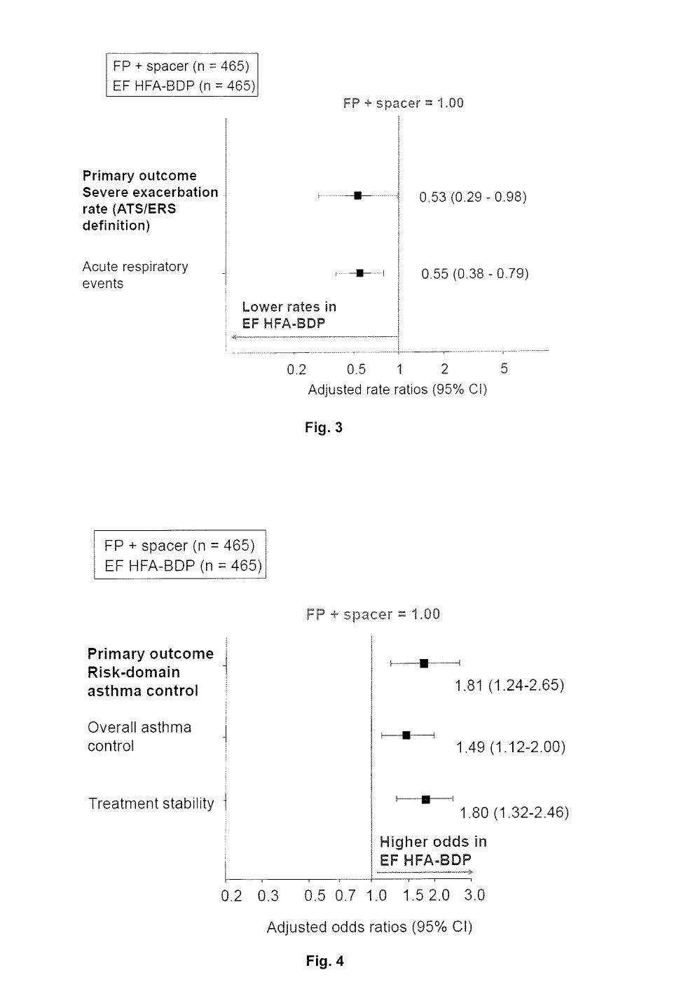

FIG. 3 shows the outcome of severe exacerbation rates in the study; and

FIG. 4 shows the odds ratios during the outcome year of the study.

The present invention will now be described with reference to the following examples, which are not intended to be limiting.

### EXAMPLE

Study Design

A study was conducted on children which compared outcomes achieved by standard particle ICS fluticasone propionate (FP) with a spacer with those achieved by the extra fine particle ICS hydrofluoroalkane beclomethasone dipropionate of the present invention (EF HFA-BDP, with or without spacer). The study design is shown in FIG. 1.

Study Population

The study population was taken from two datasets, Clinical Practice Research Datalink (CPRD): anonymised longitudinal medical records from approximately 500 primary care practices in the UK (http://www.cprd.com/intro.asp), and Optimum Patient Care Research Database (OPCRD): Anonymous longitudinal data from approximately 350 medical centres around the UK comprising a population of over 720,000 patients (http://www.optimumpatientcare.org/Html_Docs/OPCRD.html). The study population is shown in FIG. 2.

Of the patients receiving the EF HFA-BDP, 319 out of 465 (69%) were using a spacer during the outcome year. Of the patients receiving FP, 100% were using a spacer.

Outcomes

There were two co-primary outcomes. A risk-domain asthma control was defined as an absence of the following during outcome year: 1. An asthma related in-patient admission, emergency room visit, out-of-hours consultation or out-patient department attendance, 2. A GP visit for lower respiratory tract infections (LRTI) with prescription of antibiotics, or 3. An acute course of oral corticosteroids. Severe exacerbation rate (ATS/ERS definition): was defined as: 1. An asthma related in-patient or ER visit, or 2. An acute course of oral corticosteroids.

There were three secondary outcomes. Overall asthma control was defined as an absence of the following during outcome: 1. Achieving of risk-domain asthma control and, 2. An average consumed daily salbutamol-equivalent dose of <200 mg. Acute respiratory events were defined as either 1.ATS/ERS defined severe exacerbation, or 2. GP consultation for LRTI with prescription for antibiotics. Treatment stability was defined as the following during outcome year: 1. Achieving of risk-domain asthma control and, 2. No change in therapeutic regimen (Increased ICS dose or use of additional asthma therapy, defined as long-acting bronchodilator, theophylline, or leukotriene receptor antagonists).

Methods
Software: SPSS v20, SAS v9.3 and Excel 2007.
Exploratory analysis: for baseline and outcome variables:
   Treatment arms were compared using Mann-Whitney U tests (for variables measured on the interval/ratio scale) and Chi- square tests (for categorical variables).
   Exploratory analyses were used to inform decision of matching criteria for adjusted outcome analyses.
Matched baseline analysis: summary statistics produced for matched treatment arms to describe demographics and treatment in the baseline year.
Matching: EF HFA-BDP patients with or without spacer were matched 1:1 to FP patients using spacer to ensure comparison of similar patients.
Outcome Analysis:
   All multivariate models were adjusted for residual confounding factors (p<0.05 for confounding factors predictive of outcome variables, p<0.1 for residual differences in treatment arms).
   Adjusted severe exacerbation rates (ATS/ERS definition; acute respiratory events) were assessed using conditional Poisson regression models.
   Adjusted odds of achieving risk-domain asthma control, overall asthma control and treatment stability were compared using conditional binary logistic regression models.
   Results were presented as adjusted odds ratios and rate ratios with 95% confidence intervals (Cis).

Results

The baseline data is provided in Table 1:

TABLE 1
Baseline characteristics

| | | EF HFA-BDP | FP + spacer |
|---|---|---|---|
| | | n = 465 | n = 465 |
| Age* Mean (SD) | | 7.36 (1.99) | 7.36 (1.99) |
| Sex* Male n (%) | | 263 (56.6) | 263 (56.6) |
| BMI (kg/m²) Median (IQR) | | 16.4 (15.1, 18.5) | 16.1 (14.9, 18.3) |
| Rhinitis diagnosis, n (%) | | 68 (14.6) | 81 (17.4) |
| Preschool wheeze diagnosis n (%) | | 50 (15.3) | 36 (10.9) |
| ATS/ERS* | 0 | 419 (90.1) | 419 (90.1) |
| exacerbations | 1 | 42 (9.0) | 42 (9.0) |
| n (%) | 2+ | 4 (0.9) | 4 (0.9) |
| | 0 | 361 (77.6) | 364 (78.3) |
| Acute respiratory | 1 | 75 (16.1) | 75 (16.1) |
| events n (%) | 2+ | 29 (6.2) | 26 (5.6) |
| | None | 148 (31.8) | 150 (32.3) |
| Baseline asthma | SABA | 310 (66.7) | 295 (63.4) |
| therapy | Other | 7 (1.5) | 20 (4.3) |

| | | |
|---|---|---|
| Baseline use of a spacer device n (%) | 169 (36.3) | 233 (50.1) |
| Date of initiation of ICS, median (IQR) | 2005 (2003, 2007) | 2003 (2001, 2006) |

*Matching criteria

There was a significant difference between the doses of ICS across treatment arms at the date of ICS initiation (see Table 2).

TABLE 2

| | EF HFA-BDP | FP + spacer | |
|---|---|---|---|
| | n = 465 | n = 465 | P-value |
| Initial prescribed ICS | 100 (100-200) | 200 (100-200) | <0.001 |
| Dose (mcg), median | | | |
| [IQR] FP | | | |
| equivalent dose | | | |

FIG. 3 shows the outcome severe exacerbation rate ratios. The hydrofluoroalkane beclomethasone according to the invention with or without a spacer device is associated with significantly reduced rates of severe exacerbations, using both the ATS/ERS task-force and acute respiratory events definitions, compared with fluticasone propionate with a spacer device.

FIG. 4 shows the odds ratios during outcome year. Use of hydrofluoroalkane beclomethasone according to the present invention gives significantly improved odds of asthma control and treatment stability compared with fluticasone propionate with a spacer device.

The study therefore shows that children receiving hydrofluoroalkane beclomethasone dipropionate of the invention with or without a spacer had better clinical outcomes compared with children initiating fluticasone propionate with a spacer. This suggests that split-second co-ordination of inhalation in children may be less important compared with standard particle size with a fine particle inhaled corticosteroid.

What is claimed is:

1. An inhalable pharmaceutical solution aerosol comprising beclometasone dipropionate, ethanol and a propellant selected from 1,1,1,2-tetrafluoroethane, 1,1,1,2,3,3,3-heptafluoropropane and a mixture thereof, wherein the aerosol has a droplet size having a mass median aerodynamic diameter of 0.5-2.0 μm, for use in the treatment of asthma in an individual with impaired hand-inhalation coordination.

2. The inhalable pharmaceutical solution aerosol for use of claim 1, wherein the individual demonstrates either a positive or negative time difference between the start of an inhalation and actuation of a dose (TsIn) of greater than 0.2 seconds.

3. The inhalable pharmaceutical solution aerosol for use of claim 1, wherein the individual demonstrates either a positive or negative time difference between the start of an inhalation and actuation of a dose (TsIn) of greater than 0.5 seconds.

4. The inhalable pharmaceutical solution aerosol for use of claim 1, wherein the individual demonstrates either a positive or negative time difference between the start of an inhalation and actuation of a dose (TsIn) of greater than 1.0 seconds.

5. The inhalable pharmaceutical solution aerosol for use of claim 1, comprising 0.02 to 0.6 percent by weight beclometasone dipropionate, 1 to 20 percent by weight ethanol and 80 to 99 percent by weight of propellant, wherein the percentages by weight are based on the total weight of the solution aerosol.

6. The inhalable pharmaceutical solution aerosol for use of claim 1, consisting essentially of beclometasone dipropionate, ethanol and a propellant selected from 1,1,1,2-tetrafluoroethane, 1,1,1,2,3,3,3-heptafluoropropane and a mixture thereof.

7. The inhalable pharmaceutical solution aerosol for use of claim 1, wherein the aerosol has a droplet size having a mass median aerodynamic diameter of 0.8-1.2 μm.

8. The inhalable pharmaceutical solution aerosol for use of claim 1, wherein the individual does not inhale with the use of a spacer.

9. The inhalable pharmaceutical solution aerosol for use of claim 1, wherein the individual is aged from 5 to 11 years.

10. The inhalable pharmaceutical solution aerosol for use of claim 1, wherein the individual is aged from 60 years and above.

11. The inhalable pharmaceutical solution aerosol for use of claim 1, wherein the individual is suffering from arthritis.

12. A method for treating asthma in an individual with impaired hand-inhalation coordination, wherein the method comprises administering to the individual via inhalation an inhalable pharmaceutical solution aerosol comprising beclometasone dipropionate, ethanol and a propellant selected from 1,1,1,2-tetrafluoroethane, 1,1,1,2,3,3,3-heptafluoropropane and a mixture thereof, wherein the aerosol has a droplet size having a mass median aerodynamic diameter of 0.5-2.0 μm.

13. The method of claim 12, wherein the inhalable pharmaceutical solution aerosol is administered to the individual using a pressurised metered-dose inhaler.

14. The method of claim 13, wherein the pressurised metered-dose inhaler is comprised of a discharge nozzle having an orifice diameter of 100-300 μm.

15. The method of claim 12, wherein the individual does not inhale with the use of a spacer.

16. The method of claim 12, wherein the individual is aged from 5 to 11 years.

17. The method of claim 12, wherein the individual is aged from 60 years and above.

18. The method of claim 12, wherein the individual is suffering from arthritis.

19. A pressurized metered-dose inhaler useful for treatment of asthma in an individual with impaired hand-inhalation coordination, comprising a canister and an actuator having a discharge nozzle having an orifice diameter of 100-300 μm, wherein the canister has a vial containing a pharmaceutical solution comprising beclometasone dipropionate, ethanol and a propellant selected from 1,1,1,2-tetrafluoroethane, 1,1,1,2,3,3,3-heptafluoropropane or a mixture thereof, and wherein the pressurized metered-dose inhaler is configured to form, upon actuation, an inhalable aerosol of the pharmaceutical solution having a droplet size having a mass median aerodynamic diameter of 0.5-2.0 μm.

20. The pressurized metered-dose inhaler of claim 19, wherein the pharmaceutical solution is comprised of 0.02 to 0.6 percent by weight beclometasone dipropionate, 1 to 20 percent by weight ethanol and 80 to 99 percent by weight of propellant, wherein the percentages by weight are based on the total weight of the pharmaceutical solution.

21. The pressurized metered-dose inhaler of claim 19, wherein the pharmaceutical solution consists essentially of beclometasone dipropionate, ethanol and a propellant selected from 1,1,1,2-tetrafluoroethane, 1,1,1,2,3,3,3-heptafluoropropane and a mixture thereof.

22. The pressurized metered-dose inhaler of claim 19, wherein the inhalable aerosol has a droplet size having a mass median aerodynamic diameter of 0.8-1.2 μm.
